(19) United States
(12) Patent Application Publication
(10) Pub. No.: US 2014/0052239 A1
(43) Pub. Date: Feb. 20, 2014

(54) CONVEYING APPLIANCE WITH VALVE LOCATING FUNCTION FOR USE DURING PERCUTANEOUS AORTIC VALVE REPLACEMENT OPERATION

(71) Applicants: Xiangqing Kong, Nanjing (CN); Jing Cao, Wuxi (CN)

(72) Inventors: Xiangqing Kong, Nanjing (CN); Jing Cao, Wuxi (CN)

(73) Assignees: Jing CAI, Wuxi, Jiangsu (CN); Xiangqing KONG, Nanjing, Jiangsu (CN)

(21) Appl. No.: 13/990,275

(22) PCT Filed: Dec. 15, 2010
(86) PCT No.: PCT/CN2010/079798

§ 371 Date: Jul. 30, 2013

(30) Foreign Application Priority Data
Nov. 30, 2010 (CN) 201010564929.9

(51) Int. Cl. A61F 2/24

(52) U.S. Cl. CPC A61F 2/2427
USPC 623/2.11

(57) ABSTRACT

A conveying appliance with valve locating function for use during percutaneous aortic valve replacement operation includes a conveying rod (1), a sheath pipe (5), a round ring (3), at least two locating rods (2) and at least two round ring operating ropes (4). The round ring (3) is sleeved on the lower end of the conveying rod (1) and located in the conveying sheath pipe (5). An end of the locating rod (2) is hinged on the conveying rod (1) and the hinge point is located on the upper portion of the round ring (3). A lower end of the round ring operating rope (4) is provided with a structure for supporting the round ring (3) or is directly connected with the round ring (3). An upper end of the round ring operating rope (4) extends out of a human body along the axial direction of the conveying rod (1). The appliance can locate the implanted valve in a better manner so as to increase the success rate of percutaneous aortic valve replacement operation and reduce the operation difficulty and the postoperative complications. The appliance reduces the pain and the risk of the operation, and lowers the medical cost.

### FIELD OF THE INVENTION

The present invention relates to a medical appliance, especially a conveying appliance for precisely locating a valve stent during percutaneous aortic valve replacement operation, and in more particular, to a conveying appliance with valve locating function for use during percutaneous aortic valve replacement operation.

### BACKGROUND OF THE INVENTION

At present, the statistical data from the related art show that the disease incidence of aortic valve stenosis attributed to aortic valve calcification has reached 2% to 7% among elderly people over 65 years old, and is becoming increasingly higher with age. Usually, patients with serious aortic valve stenosis are heavily insufficient of left ventricular function, which is accompanying with decreased living quality and shortened lifespan, thus requires effective treatment. Until now, artificial aortic valve replacement by means of surgical operation is still the preferred treatment for patients subjected to indications.

The word's first transcatheter artificial aortic valve replacement is conducted on a 57-year old patient with serious aortic valve stenosis by Alain Cribier in 2002, based on a large number of animal experiments. Since then, the transcatheter aortic valve replacement, advantageous in small surgical trauma and less postoperative complications, brings hope to the patients who have clinically lost opportunities of surgical procedures, and has been improving and developing in many countries. Currently, the widely popular one of the transcatheter aortic valve replacements is the aortic valve implantation (AVI) with self-expanding trifoliate valve, which is continuously improved in terms of biological materials and conveying appliances, thus makes the percutaneous aortic valve replacement becoming possible.

One of the factors that hinder the development of the percutaneous aortic valve replacement technology is the difficult control during locating the appliances for implantation, it may result in dropping of the instruments when released or results in inaccurate positioning, which will heavily influence the success rate of the procedure and the control of postoperative complications.

### SUMMARY OF THE INVENTION

The present invention intends to overcome the problem of incapable of adjusting the valve position during the existing valve replacement which is liable to result in failure of operation or result in postoperative complications due to valve dislodgement. The above objective is achieved by a conveying appliance with valve locating function for use during percutaneous aortic valve replacement operation. According to the present invention, said conveying appliance capable of inserting into human body along with a valve conveying appliance not only can prevent from valve stent dislodgement when released, but also can adjust the position of the valve stent.

In particular, the conveying appliance with valve locating function for use during percutaneous aortic valve replacement operation comprising:

a conveying rod 1, one end of the conveying rod 1 that is inserting into the human body is used for sheathing (sleeving onto) a valve stent 9;
a sheath pipe 5 that is sleeved on the conveying rod 1 and used for receiving the valve stent 9;
a round ring 3 that is sleeved on the lower end of the conveying rod 1 for use during percutaneous aortic valve replacement operation and is located in the sheath pipe 5;
at least two locating rods 2, one end of the locating rod 2 is hinged on the conveying rod 1 and the hinge point is located on the upper portion of the round ring 3; and
at least two round ring operating ropes 4, an lower end of the round ring operating rope 4 is provided with a structure for supporting the round ring 3 or is directly connected with the round ring 3, and an upper end of the round ring operating ropes 4 is extending out of the human body along an axial direction of the conveying rod 1.

The above-mentioned conveying rod 1, sheath pipe 5, round ring 3, locating rod 2 and round ring operating rope 4, together, constitute the conveying appliance with valve locating function for use during percutaneous aortic valve replacement operation of the present invention.

A cantilever end of the locating rod 2 is provided with a round arc structure or a round ring structure; the locating rod 2 is capable of stretching within a range of at least 90 degree; and the length of the body of the locating rod 2 shall be longer than the radius of the left ventricular outflow tract (LVOT).

The structure for supporting the round ring 3 at the lower end of the round ring operating rope 4 is a supporting transverse rod which is perpendicular to the main body of the round ring operating rope 4; the supporting transverse rod 6 and the round ring operating rope 4 constitute an L-shape structure together.

The surface of the conveying rod 1 is provided with axial grooves 7, at an amount equal to that of the round ring operating ropes 4.

The end of the round ring operating ropes (4) that is extending out of the human body is connected with a conventional, synchronously or individually manipulating mechanism.

The present invention is advantageous in that:

i. it can assist with the locating of the implanted valve in an excellent manner, so as to increase the success rate of percutaneous aortic valve replacement operation, reduce the operation difficulty, relieve the pain of the patients, and lower the risk of the procedure and the medical cost;
ii. it's no need of increasing the inner diameter of the conveying sheath pipe, because all of the locating appliances can be folded into thick pipes;
iii. the respective portions as well as the orientation of the valve after releasing can be slightly adjusted by the locating appliance of the present invention, which optimizes the implantation position;
iv. the tail end of the locating rod is provided with a hook-like structure, which can prevent certain tissues such as bundle branch and chordae tendineae of bicuspid valve from being subjected to compression, so as to reduce postoperative complications and improve living quality of the patients after procedure;
v. the invention is simple in structure, convenient in manufacture and easy to implement by appropriate modification to the conveying rod of prior art.

### BRIEF DESCRIPTION OF THE DRAWINGS

FIG. 1 is a schematic view showing the structure of the locating appliance according to the present invention when folded;

FIG. 2 is a schematic view showing the state of the locating appliance according to the present invention when using in human body;

FIG. 3 is a schematic view showing the cross section of the conveying rod according to the present invention;

in which,
9: implanted valve;
10: coronary sinus of the human body; and
11: left ventricular outflow tract of the human body.

### DETAILED DESCRIPTION OF THE INVENTION

Hereinafter the present invention will be described in details with reference to the drawings and embodiment.

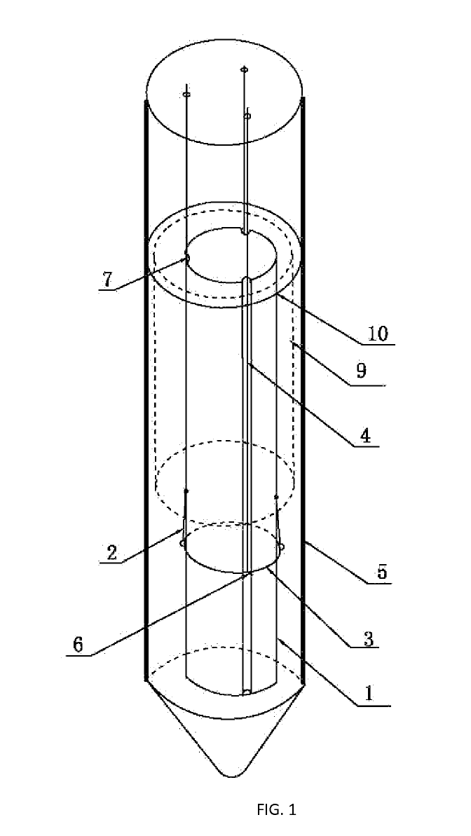

Please refer to FIG. 1-FIG. 3.

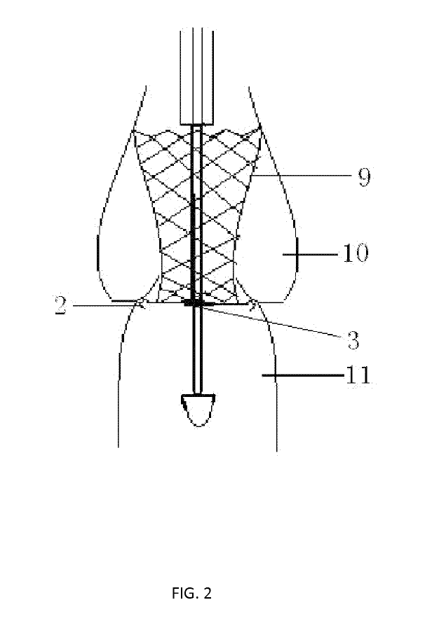

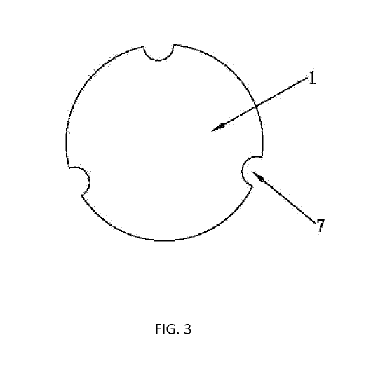

The conveying appliance with valve locating function for use during percutaneous aortic valve replacement operation mainly comprises conveying rod 1, sheath pipe 5, locating rod 2, round ring 3 and round ring operating rope 4. As shown in FIG. 1, the conveying rod 1 is hinged on the distal end (the end located in the human body, i.e., the lower end) of the conveying rod 1 by means of fixed hinge; the tail end of the locating rod 2 is designed as round arc-shaped or round ring-shaped hook so as to prevent from injuring the human body due to a sharp tip; the cross section of the rod body of the locating rod 2 is in a shape of round arc with smooth edge; the assembled hook-like portion shall have a diameter smaller than the inner diameter of the conveying sheath pipe 5; the locating rods 2 are capable of stretching (like opening up an umbrella) within a range of at least 90 degree, driven by the round ring 3; the length of the rod body of the locating rod 2 shall be longer than the radius of the left ventricular outflow tract of the implanted valve 9; the state of the stretched locating rod 2 is shown as FIG. 2, in which the diameter generated from the stretched locating rod 2 is larger than the outer diameter of the outflow tract of the implanted valve, thus the valve being released will fall onto the locating rod 2 at the first place, without dropping onto other undesirable places. When assembled, the round ring 3 is located in the hook-like structure of the locating rod 2 such that the round ring 3 allows the locating rods 2 stretching like umbrella ribs when the round ring 3 is pulled to move upwards; the stretched locating rods 2 then can slide on the conveying rod 1 so as to adjust the position and orientation of the valve stent; the tail end of the round ring operating rope 4 may be L-shaped, and the transverse rod 6 perpendicular to the main body of the round ring operating rope 4 is mainly functioned for hooking the round ring 3, for the convenience of adjustment and control, and the convenience of releasing the round ring 3; the lower end of the round ring operating rope 4 is provided with an L-shaped structure for supporting the round ring 3, and the upper end of the round ring operating rope 4 is penetrating through the axial groves 7 (see FIG. 3) on the conveying rod 1 and the through holes (located on the upper portion of a segment of the conveying rod 1, which segment is sleeved onto the valve stent 9, i.e., the step-shaped portion 10 in FIG. 3) and then extending out of the human body to connect to a manipulating mechanism, which is usually a conventional, synchronous, draw-off mechanism; that is to say, the upper ends of a plurality of round ring operating ropes 4 are fixed onto a draw-off block at the same time, and the draw-off block is then pulled by human hands or pulled electrically so as to drive the round ring 3 to move, thus when a slight adjustment is required, each of the round ring operating ropes 4 can be individually manipulated. In addition, when implemented, the lower end of the round ring operating ropes 4 may be directly fixed to the round ring 3 or connected to the round ring 3 by sleeve connection.

The state of the locating appliance according to the present invention when not used is shown as FIG. 1, in which the locating appliance is encapsulated within the sheath pipe 5. When used, the round ring operating ropes 4 are pulled from outside (outside of the human body) so as to drive the round ring 3 moving upwards, which in turn drives the locating rods 2 to stretch; at the same time, the entire conveying appliance is pulled upwards after the valve is released with two-thirds, then the tail end of the locating rod, with a hook-like structure, presses against the upper edge of the outflow tract, so as to release the remaining one-third of valve, as shown in FIG. 2. The position and orientation of the implanted valve can be slightly adjusted by pulling the round ring operating ropes 4. When loosing the round ring operating ropes 4 to make the round ring 3 sliding downwards into the hook-like structure of the locating rod 2, the sheath pipe 5 is advancing forwards to receive the locating rod 2 and the round ring 3 therein, the latter then are withdrawn. The whole interventional operation is conducted under the guidance of transesophagel echocardiography, which is combined with the radiography to evaluate the position of the implanted valve, the influences to coronary blood flow and conduction tract, and the occurrence of valve regurgitation.

Hereinafter the present invention will be further described in conjunction with particular animal experiments.

The experiments are carried out under the following conditions:

Five sheep, each has a weight of 45-50 kg, with an average weight of 46.8 kg.

The whole interventional operation is conducted under X-ray scan and the guidance of transesophagel echocardiography. The experimental animal is lying on its back and fixed onto a catheter surgical table. An intubation is performed and the tube is connected to a breathing machine and an ECG machine, after anesthetized with ketamine. The right carotid artery is punctured to introduce the 18F arterial sheath. Conduct left ventricle angiography with 90 degree at a left-lateral position, to obtain the inner diameters of the left ventricular outflow tract and the aorta. A percutaneous aortic valve replacement appliance of the present invention, with a diameter larger than the inner diameter of the aorta by 10%-20%, is conveyed into the human body, by using a conveying system; and the aortic valve is released and implanted at position, with the assistance of the locating appliance. Observe the positions of the appliances for implantation, by combining cardiac ultrasound technology and aortic root radiography, to evaluate valve regurgitation and coronary blood flow; and evaluate the influences to the conduction tracts by using electrocardiogram. When it's confirmed that no valve regurgitation is occurred and that the coronary blood flow and electrocardiogram are normal, the surgery is completed.

The experiments demonstrated that four of five sheep survived and one was dead from excessive bleeding during operation. The surviving animals were sacrificed at 2, 4 and 8 weeks after operation, respectively, with electrocardiographic examination in advance which represents normal aortic valve flow and no aortic valve regurgitation. With general observation of the sacrificed animals, all the percutaneous aortic valve replacement appliances are in position without any dislodgement. With visual inspection, there was no thrombus occurred on or around the surface of the percutaneous aortic valve replacement appliance. At 4 weeks after operation, 80% of the surface of the percutaneous aortic valve replacement appliance has been covered by endothelium; and at 8 weeks after operation, almost the entire surface of the percutaneous aortic valve replacement appliance was covered by endothelium.

The description above only illustrates part of the embodiments of the present invention. It should be pointed out that, various modifications and polishes may be made by a person skilled in the art without departing from the principle of the present invention. These modification and polishes shall also be regarded as the extent of protection of the present invention.

What is claimed is:

1. A conveying appliance with valve locating function for use during percutaneous aortic valve replacement operation, comprising:
a conveying rod (1), one end of the conveying rod (1) that is inserting into the human body is used for sheathing a valve stent (9);
a sheath pipe (5) that is sleeved on the conveying rod (1) and used for receiving the valve stent (9);
a round ring (3) that is sleeved on the lower end of the conveying rod (1) for use during percutaneous aortic valve replacement operation and is located in the sheath pipe (5);
at least two locating rods (2), one end of the locating rod (2) is hinged on the conveying rod (1) and the hinge point is located on an upper portion of the round ring (3); and
at least two round ring operating ropes (4), an lower end of the round ring operating rope (4) is provided with a structure for supporting the round ring (3) or is directly connected with the round ring (3), and an upper end of the round ring operating rope (4) is extending out of the human body along an axial direction of the conveying rod (1).

2. The conveying appliance with valve locating function for use during percutaneous aortic valve replacement operation according to claim 1, wherein a cantilever end of the locating rod (2) is provided with a round arc structure or a round ring structure; the locating rods (2) are capable of stretching within a range of at least 90 degree; and the length of the rod body of the locating rod (2) is longer than the radius of the left ventricular outflow tract (LVOT) of the implanted valve stent.

3. The conveying appliance with valve locating function for use during percutaneous aortic valve replacement operation according to claim 1, wherein the structure for supporting the round ring (3) at the lower end of the round ring operating rope (4) is a supporting transverse rod (6) which is perpendicular to the main body of the round ring operating rope (4); the supporting transverse rod (6) and the round ring operating rope (4) constitute an L-shape structure together.

4. The conveying appliance with valve locating function for use during percutaneous aortic valve replacement operation according to claim 1, wherein the surface of the conveying rod (1) is provided with axial grooves (7), at an amount equal to that of the round ring operating ropes (4).

5. The conveying appliance with valve locating function for use during percutaneous aortic valve replacement operation according to claim 1, wherein the end of the round ring operating ropes (4) that is extending out of the human body is connected with a synchronously or individually manipulating mechanism.
